(19) United States
(12) Patent Application Publication
(10) Pub. No.: US 2022/0340870 A1
(43) Pub. Date: Oct. 27, 2022

(54) FUNCTIONAL NEUROMODULATORY ASSEMBLOIDS

(71) Applicant: The Board of Trustees of the Leland Stanford Junior University, Stanford, CA (US)

(72) Inventors: Sergiu P. Pasca, Menlo Park, CA (US); Fikri Birey, Palo Alto, CA (US)

(21) Appl. No.: 17/641,710

(22) PCT Filed: Sep. 10, 2020
(86) PCT No.: PCT/US2020/050252

§ 371 Date: Mar. 9, 2022

Related U.S. Application Data
(60) Provisional application No. 62/898,430, filed on Sep. 10, 2019.

(51) Int. Cl. C12N 5/0793; G01N 33/50

(52) U.S. Cl. CPC C12N 5/0619; G01N 33/5058; C12N 2506/45; C12N 2513/00; C12N 2501/155; C12N 2501/15; C12N 2501/727; C12N 2501/119; C12N 2501/41; C12N 2501/13; C12N 2501/01

(57) ABSTRACT

Human raphe nuclei organoids or spheroids (hRNS) are generated in vitro, which may be generated at least in part from human pluripotent stem (hPS) cells. Such spheroids model the human raphe nuclei and comprise specific sets of cells, e.g. serotonergic neurons, that are associated with the raphe nuclei of a human, and can be assembled with cortical spheroids (hCS) to generate functional human neuromodulatory circuits.

### CROSS REFERENCE

This application claims priority to U.S. Provisional Application No. 62/898,430, filed Sep. 10, 2019 which is incorporated herein in its entirety for all purposes.

### BACKGROUND OF THE INVENTION

The mammalian neuromodulatory system consists of discrete groups of neurons that project from the brainstem, pontine nucleus, or basal forebrain, which fine tune brain function and have been implicated in various neuropsychiatric disorders. Neuromodulators such as serotonin, norepinephrine, acetylcholine, dopamine can operate at multiple timescales on these cells, ranging from short-term adjustments of neuron and synapse function to long-term circuit adaptations. During early brain development, neuromodulators actively participate to the assembly of the nervous system by modulating cell division, differentiation, migration, synaptogenesis, synapse transmission and dendritic pruning. One of the earliest neuromodulatory innervations into the developing cerebral cortex is via serotonergic neurons that originate from raphe nuclei located in the midline of the brainstem.

The serotonin (5HT) system, despite consisting of only about half a million neurons in the human brain, innervates nearly every region of the central nervous system. Serotonin acts through at least fourteen different G protein-coupled receptors, which have been shown to be divergent in the human cerebral cortex versus other mammals and primates. Depending on the subtype, these receptors can exert either inhibitory or excitatory modulatory neuronal activity.

In the human central nervous system, serotonergic neurons are generated as early as 5 weeks post-conception and by 15 post-conception weeks, raphe nuclei already contain a stereotypical arrangement of serotonin neurons. Importantly, malfunction of serotonergic neurotransmission has long been associated with major depressive disorder (MDD), and many of the pharmacological agents used in MDD, such as selective serotonin reuptake inhibitors (SSRIs), work by modulating this pathway. SSRIs are the first line of treatment for MDD. However, a significant percentage of patients remain SSRI-resistant and it is unclear whether and how alterations in serotonergic neurons contribute to SSRI resistance in these patients. Additionally, serotonin signalling has been implicated in multiple other neuropsychiatric disorders, such as schizophrenia, affective disorders, anxiety, and autism spectrum disorder.

Region-specific brain organoids or brain spheroids to not currently contain neuromodulatory systems and there are no human platforms to study, manipulate or investigate neuromodulatory pathways with human patient derived cells.

### SUMMARY OF THE INVENTION

Compositions and methods are provided for in vitro generation of human raphe nuclei organoids or spheroids (hRNS), which may be generated at least in part from human pluripotent stem (hPS) cells. Such spheroids model the human raphe nuclei and comprise specific sets of cells, e.g. serotonergic neurons, GABAergic neurons, etc that are associated with the raphe nuclei of a human.

The hRNS can be functionally integrated with human cerebral cortical spheroids (hCS), which spheroids comprise human cortical neurons, such as glutamatergic neurons, to provide a cortico-raphe nuclei assembloid (hCS-hRNS). Human serotonergic neurons form bidirectional projections between neurons of the hRNS and hCS to generate a neuromodulatory assembloid. This assembloid is comprised of functionally integrated cells, including neurons, which interact in a physiologically relevant manner, e.g. forming synapses between classes of neurons so as to provide physiologically relevant, functional neural circuits. Using a combination of viral tracing and live imaging, evidence is provided herein for the formation of an in vitro generated human cortico-raphe nuclei neural circuit, which provides useful modeling of cortico-raphe nuclei pathway development and dysfunction.

In some embodiments, assembloids are provided in which one or more of the cells have been genetically modified to provide for additional functionality in screening. For example, one or both of cortical neurons and serotonergic neurons can be genetically modified to express a fluorescent calcium indicator, which indicators are known and used in the art. One or both of cortical neurons and serotonergic neurons can be genetically modified to express a light activated opsin. In some embodiments, an assembloid comprises serotonergic neurons expressing an opsin, and cortical, e.g. glutaminergic, neurons expressing a fluorescent calcium indicator, where a functional relationship between the neurons is demonstrated by using light to activate the serotonergic neuron, and observing a calcium indicator response from a cortical neuron. 5HT lineage-specific viral tools, such as AAV-based FEV minipromoter-driven reporters, are available and have been shown to work below to specifically probe and study 5HT lineage cells in hRNS and hCS-hRNS.

The hRNS spheroid and hCS-hRNS assembloid provide unique opportunities for analysis of the development and function of serotonergic neural circuits between the raphe nuclei and the cortex (and viceversa) and serotonergic modulation of cortical neural circuits. Further, these spheroids or brain-region specific organoids and assembloids provide a model to study the impact neurologic or psychiatric disorders have on neural circuits in these brain regions. Of particular relevance are those neurologic or psychiatric disorders that are associated with serotonin dysfunction, such as MDD, schizophrenia and other psychoses, affective disorders (e.g. depression, bipolar disorder or an anxiety disorder) and autism spectrum disorder (ASD).

Additionally, these spheroids and assembloids can be used to establish a screening platform for SSRI function and, for example, model different pharmacological modulation by SSRIs and atypical antipsychotics on the serotonergic system, analyse serotonergic-related disorders such as serotonin syndrome, analyse the in utero effects of SSRIs on cortical development, etc. For example, in a neuromodulatory assembloid system where cortical neuron activity is monitored with a calcium indicator (gCamp6 or Fluo-4) or a voltage indicator and/or the activity of serotonergic neurons is modulated with an electrode, optogenetics, etc, the system can be utilized in high-throughput assays for libraries of candidate agents, such as modulators of 5-HT receptors (e.g., antipsychotics) or 5-HT transporters (SSRI) to test their relative physiological effects (i.e., calcium amplitude, calcium spike frequency, voltage changes of the neuronal membrane, etc) as compared to drugs of known activity. The models provided herein also allow testing the effect of the genetic background, e.g. by using multiple assembloids from different human participants, which optionally comprise gene variants influencing the function of receptors or transporters of interest. The composition of 5HT receptors expressed by the postsynaptic neurons, which changes with neuronal subtype and developmental age, determines the effect of stimulation and pharmacological application in this system, resulting in altered neuromodulatory responses such as prolonged inhibition or excitation, tonic, adapting, bursting modes of firing. Comparison to one or more known control agent(s) may be used to determine the desired response.

This system additionally has the advantage of providing an opportunity of using patient-derived hiPS cells. This can enable screening approaches towards elucidating mechanisms of SSRI resistance in hRNS-hCS assembloids derived from patients suffering from neurologic or psychiatric disorder (e.g. Major Depressive Disorder (MDD)) with or without SSRI resistance. Furthermore, assembloids can be generated combining control and patient cells (e.g. control-hCS with patient-hRNS) to dissect cell-autonomous contributions. This platform can also be used to study genetic forms of autism spectrum disorder linked to disturbances in 5-HT system, such as 16p11.2 microdeletion or duplication and rare, disease-causing mutations in the FEV gene.

Also provided are methods for determining the activity of a candidate agent neural circuits in the assembloids, the method comprising contacting the candidate agent with the assembloid. The cells present in the assembloid optionally comprise at least one allele encoding a mutation associated with, or potentially associated with, a neurologic or psychiatric disorder, and determining the effect of the agent on morphological, genetic or functional parameters, including without limitation neuron number, neuron function, gene expression profiling, cell death, single cell gene expression (RNA-seq), calcium imaging with pharmacological screens, patch-clamp recordings, modulation of synaptogenesis, and the like.

These and other objects, advantages, and features of the invention will become apparent to those persons skilled in the art upon reading the details of the subject methods and compositions as more fully described below.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIG. 1A-1G. (A) Schematic showing hRNS-hCS assembly (B) Schematic showing the recipe for deriving human raphe nuclei-like spheroids (hRNS). (C) RT-qPCR profiling of genes expressed in the developing hindbrain in human cortical spheroids (hCS) versus hRNS. Different hiPSC lines are represented in different colors. (D) Representative images of immunocytochemistry (ICC) showing hindbrain progenitors and (E) 5-HT neural lineage cells. (F) 5-HT neurotransmitter levels measured by HPLC from hRNS across different in vitro stages and hiPSC lines. (G) RT-qPCR profiling of different 5-HT receptors (HTRs) in hCS at day 100 of in vitro differentiation. HTR subtype is indicated by color.

FIG. 2A-2C (A) UMAP projections of single cell transcriptomic data from 13,708 hRNS cells harvested from 9 spheroids derived from 3 hiPSC lines between day 79 and 82 of in vitro differentiation. (B) Cluster marker expression showing different neuronal populations in hRNS. (C) FEV+ subclustering showing subcluster separated of caudal and rostral identities (top) and heatmap of top 10 differentially expressed genes in each cluster (bottom).

FIG. 3A-3C. (A) Assembly of hCS and AAV-Syn1::mCherry-labeled hRNS (Left). 3D reconstruction of a hCS-hRNS assembloid, with hRNS cells labeled in mCherry (Right). (B) Representative ICC images showing NKX6-1⁺ hindbrain progenitors and TPH2⁺ 5-HT expressing cells labeled in a hRNS-hCS assembloid. (C) Assembly of AAV-Syn1::mCherry-labeled hCS and AAV-Syn1::eYFP-labeled hRNS (Left). Live imaging of double-infected hRNS-hCS showing bidirectional projections (Right). Inset shows axonal morphology with spindle-like varicosities that are often seen in forebrain—projecting 5-HT⁺ neuronal processes.

FIG. 4A-4C. (A) Schematic (left), representative immunocytochemistry images (center) and co-localization quantification (right) showing the characterization of 5HT lineage-specific FEV reporter Ple67 on dissociated hRNS cells. (B) Assembly of hCS labeled with AAV-SYN1::mCherry-labeled hCS and hRNS labeled with AAV-Ple67iCRE and AAV-EF1α-DIO-eYFP (Left). Live imaging of infected hRNS-hCS showing projections of 5HT lineage cells towards hCS (Right). (C) Assembly of hCS labeled with AAV-Syn1::GCaMP7s-labeled hCS and hRNS labeled with AAV-Ple67iCRE and AAV-EF1α-DIO-ChRmine-K_{V}2.1-mScarlet (left) and example traces of optically evoked calcium transients in hCS neurons induced by 625 nm photo-stimulation of 5-HT-lineage FEV⁺ cells in hRNS (right).

The invention is best understood from the following detailed description when read in conjunction with the accompanying drawings.

### DETAILED DESCRIPTION OF THE INVENTION

Before the present compositions and methods are described, it is to be understood that this invention is not limited to particular compositions and methods described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a reprogramming factor polypeptide” includes a plurality of such polypeptides, and reference to “the induced pluripotent stem cells” includes reference to one or more induced pluripotent stem cells and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Definitions

By “pluripotency” and pluripotent stem cells it is meant that such cells have the ability to differentiate into all types of cells in an organism. The term “induced pluripotent stem cell” encompasses pluripotent cells, that, like embryonic stem (ES) cells, can be cultured over a long period of time while maintaining the ability to differentiate into all types of cells in an organism, but that, unlike ES cells, are derived from differentiated somatic cells, that is, cells that had a narrower, more defined potential and that in the absence of experimental manipulation could not give rise to all types of cells in the organism. hiPS cells have a human ES-like morphology, growing as flat colonies with large nucleo-cytoplasmic ratios, defined borders and prominent nuclei. In addition, hiPS cells express several pluripotency markers known by one of ordinary skill in the art, including but not limited to alkaline phosphatase, SSEA3, SSEA4, Sox2, Oct3/4, Nanog, TRA160, TRA181, TDGF 1. Dnmt3b, FoxD3, GDF3, Cyp26a1, TERT, and zfp42. In addition, the hiPS cells are capable of forming teratomas. In addition, they are capable of forming or contributing to ectoderm, mesoderm, or endoderm tissues in a living organism.

As used herein, “reprogramming factors” refers to one or more, i.e. a cocktail, of biologically active factors that act on a cell to alter transcription, thereby reprogramming a cell to multipotency or to pluripotency. Reprogramming factors may be provided to the cells, e.g. cells from an individual with a family history or genetic make-up of interest for heart disease such as fibroblasts, adipocytes, etc.; individually or as a single composition, that is, as a premixed composition, of reprogramming factors. The factors may be provided at the same molar ratio or at different molar ratios. The factors may be provided once or multiple times in the course of culturing the cells of the subject invention. In some embodiments the reprogramming factor is a transcription factor, including without limitation, Oct3/4; Sox2; Klf4; c-Myc; Nanog; and Lin-28.

Somatic cells are contacted with reprogramming factors, as defined above, in a combination and quantity sufficient to reprogram the cell to pluripotency. Reprogramming factors may be provided to the somatic cells individually or as a single composition, that is, as a premixed composition, of reprogramming factors. In some embodiments the reprogramming factors are provided as a plurality of coding sequences on a vector. The somatic cells may be fibroblasts, adipocytes, stromal cells, and the like, as known in the art. Somatic cells or hiPS cells can be obtained from cell banks, from normal donors, from individuals having a neurologic or psychiatric disease of interest, etc.

Following induction of pluripotency, hiPS cells are cultured according to any convenient method, e.g. on irradiated feeder cells and commercially available medium. The hiPS cells can be dissociated from feeders by digesting with protease, e.g. dispase, preferably at a concentration and for a period of time sufficient to detach intact colonies of pluripotent stem cells from the layer of feeders. The spheroids can also be generated from hiPS cells grown in feeder-free conditions, by dissociation into a single cell suspension and aggregation using various approaches, including centrifugation in plates, etc.

Genes may be introduced into the somatic cells or the hiPS cells derived therefrom for a variety of purposes, e.g. to replace genes having a loss of function mutation, provide marker genes, etc. Alternatively, vectors are introduced that express antisense mRNA, siRNA, ribozymes, etc. thereby blocking expression of an undesired gene. Other methods of gene therapy are the introduction of drug resistance genes to enable normal progenitor cells to have an advantage and be subject to selective pressure, for example the multiple drug resistance gene (MDR), or anti-apoptosis genes, such as BCL-2. Various techniques known in the art may be used to introduce nucleic acids into the target cells, e.g. electroporation, calcium precipitated DNA, fusion, transfection, lipofection, infection and the like, as discussed above. The particular manner in which the DNA is introduced is not critical to the practice of the invention.

Disease-associated or disease-causing genotypes can be generated in healthy hiPS cells through targeted genetic manipulation (CRISPR/Cas9, etc.) or hiPS cells can be derived from individual patients that carry a disease-related genotype or are diagnosed with a disease. Moreover, neural and neuromuscular diseases with less defined or without genetic components can be studied within the model system. A particular advantage of this method is the fact that edited hiPS cell lines share the same genetic background as their corresponding, non-edited hiPS cell lines. This reduces variability associated with line-line differences in genetic background. Conditions of neurodevelopmental and neuropsychiatric disorders and neural diseases that have strong genetic components or are directly caused by genetic or genomic alterations can be modeled with the systems of the invention.

The methods and compositions described herein are associated with brain-region specific spheroids. Brain-region specific spheroids are three-dimensional (3D) aggregates of cells that resemble particular regions of the human brain and contain functional neurons that are normally associated with that region of the brain. These spheroids are capable of being maintained in suspension culture for long periods of time, e.g. 2 week, 4 weeks, 1 month, 2 months, 3 months, 4 months, 5 months, 6 months or more, without adhering to a surface, e.g. a surface of a culture dish. By functional neurons, it is intended to mean that the neurons are capable of forming functional synapses with other neurons, either in the same spheroid or in another spheroid. The formation of functional synapses can be revealed using calcium imaging, as described in more details in the Examples. For example, the human raphe nuclei spheroids described herein comprise raphe nuclei neurons, such as serotonergic neurons.

The methods and compositions described herein are also associated with assembloids comprising more than one (e.g. two or three or more) of these brain-region specific spheroids. The assembloids described herein resemble multiple regions of the brains and contain functional neural circuits between neurons of one spheroid (representing one region) and another spheroid (representing another region). For example, the cortico-raphe nuclei assembloids resemble the cortex and raphe nuclei of the human brain and contain neurons (e.g. serotonergic neurons) with projections between the raphe nuclei spheroid and cortical spheroid, where these neurons are able functionally synapse with human cortical neurons (e.g. glutamatergic neurons) of the cortical spheroid and modulate the activity of neural circuits in the cortical spheroid. Similar to the spheroids, these assembloids are also capable of being maintained for long periods of time without adhering to a surface.

Raphe Nuclei. The human raphe nuclei are a cluster of neurons located within the brain stem. A large proportion of the neurons that originate in the raphe nuclei are serotonergic neurons which project into multiple locations of the human central nervous system, including the cortex, ventral striatum, hippocampus and amygdala of the forebrain. The raphe nuclei also receive connections from the cerebral cortex and other brain regions. Through interaction with these regions and other neuromodulatory systems, serotonin influences a broad range of functions such as reward assessment, impulsivity, harm aversion and anxious states. Impairment of these systems have been linked to a variety of neuropsychiatric disorders, some of which are described in more detail below.

Serotonergic neurons are neurons that produce the neurotransmitter serotonin, also known as 5-hyroxytyptamine or 5-HT. The presence of these neurons can be detected for example by expression of serotonin, enzymes involved in the serotonin pathway, such as tryptophan 5-hydroxylase 2 (TPH2), and/or markers of mature serotonin neurons such as vesicular monoamine transporter 2 (VMAT2) and serotonin reuptake transporter (SERT). Serotonin acts though at least fourteen different G protein-coupled receptors that, depending on their subtype, can exert either excitatory or inhibitory neuronal activity.

The disclosure herein provides in vitro spheroid structures (also known as region-specific organoids) and assembloids derived therefrom that comprise serotonergic neurons. The presence of serotonergic neurons can be verified by determining the presence of neurons expressing the markers indicated above, and by the presence of serotonin produced by these neurons. An hRNS may comprise at least 1% serotonergic neurons defined by these markers as a percentage of the total cell population, at least 5%, at least 10%, at least 15%, at least 20%, at least 25% or more.

When fused hRNS with a hCS to generate an assembloid, the structure provides a model for serotonergic (5-HT) modulation of cortical circuits. The functional integration of serotonergic neurons and cortical neurons can be verified microscopically by the presence of bidirectional axonal projections by which axons of hCS-derived neurons project to hRNS and axons of hRNS-derived neurons project to hCS. Bidirectional axonal projections in hRNS-hCS can be visualized by labeling hRNS and hCS with neuron-specific viral reporters (e.g. AAV-DJ-hSyn1::mCherry for hCS and AAV-DJ-hSyn1::eYFP for hRNS) before assembly and monitoring the emergence of projections with long-term confocal imaging. A subset of these projections is expected to make synaptic connections. Of note, in addition to synaptic connections, some serotonergic neurons release 5-HT in a diffuse manner in the absence of a closely apposed postsynaptic site such that cells distal to the release site may bind the released 5-HT (termed ‘volume transmission’). Therefore, true measure of serotonergic connectivity on to hCS can also involve non-junctional neurotransmitter transmission.

Functional assays for integration of circuits may include, for example, determining signal transmission between classes of neurons; and may include determining an effect of a neuromodulatory system on modulation of cell division, differentiation, migration, synaptogenesis, and dendritic pruning. For example, in an optogenetics system, photo-stimulation of a serotonergic neuron can reveal patterns of response in functionally integrated cortical neurons, e.g. increased calcium activity in response to photo-stimulation, reduced calcium activity in response to photo-stimulation either transiently or during the entire post-stimulation period; etc. There is a variety of responses that can indicate functional connectivity and SSRI responsivity in hRNS-hCS (as described above). For example, calcium responses following stimulation can be determined, where the number of activated neurons per assembly may be 10 or more, 100 or more 103, or more. Use of a two-photon (2P) system with an optimized imaging apparatus may be used to capture higher numbers of events.

Cerebral cortex. The adult cerebral cortex contains two main classes of neurons: glutamatergic cortical neurons (also known as pyramidal cells) and GABAergic interneurons.

Glutamatergic neurons. The mature cerebral cortex harbors a heterogeneous population of glutamatergic neurons, organized into a highly intricate histological architecture. So-called excitatory neurons are usually classified according to the lamina where their soma is located, specific combinations of gene expression, by dendritic morphologies, electrophysiological properties, etc. GABAergic interneurons are inhibitory neurons of the nervous system that play a vital role in neural circuitry and activity. They are so named due to their release of the neurotransmitter gamma-aminobutyric acid (GABA). An interneuron is a specialized type of neuron whose primary role is to modulate the activity of other neurons in a neural network. Cortical interneurons are so named for their localization in the cerebral cortex.

Disease relevance. Dysfunction in serotonergic neural circuits has been associated with various neurological and psychiatric disorders including schizophrenia, affective disorder and autism spectrum disorder (ASD). The systems described here provide unique opportunities to study the role of these circuits in these disorders and allow for the screening of potential therapeutics.

Affective disorders are a set of psychiatric disorders also called mood disorders, which include depression, bipolar disorder and anxiety disorder. Altered serotonin activity has been associated with various mood disorders and selective serotonin reuptake inhibitors (SSRIs) are frequently used as treatments for affective disorders such as major depressive disorder (MDD). The underlying role of serotonin in affective disorders has not been fully elucidated and therefore the systems described here provide opportunities to further study its role in these disorders, screen for potential new SSRIs and model interactions between SSRIs and serotonergic neurons. Extremely high levels of serotonin can cause a condition known as serotonin syndrome, with toxic and potentially fatal effects, which can also be further investigated with the systems described here.

Schizophrenia. Schizophrenia is a chronic and severe mental disorder that affects an individual's behavior. The underlying cause of schizophrenia is still unclear, but the disorder has been associated with abnormal serotonin and dopamine signaling in the central nervous system. The systems described here provide opportunity to further study the role of serotonin in schizophrenia and develop potential therapeutic treatments.

Autism spectrum disorder (ASD) is a developmental disorder that affects communication and behavior and is associated. Elevated whole blood serotonin was the first biomarker identified in ASD and is present in more than 25% of affected children, although the contribution of the serotonin system to ASD pathophysiology remains incompletely understood (Muller et al. “The serotonin system in autism spectrum disorder: from biomarker to animal models” Neuroscience 321: 24-41 (2016)). The systems described here provide opportunities to study the role of serotonin in ASD.

Calcium sensors. Neural activity causes rapid changes in intracellular free calcium, which can be used to track the activity of neuronal populations. Art-recognized sensors for this purpose include fluorescent proteins that fluoresce in the presence of changes in calcium concentrations. These proteins can be introduced into cells, e.g. hiPS cells, by including the coding sequence on a suitable expression vector, e.g. a viral vector, to genetically modify neurons generated by the methods described herein. GCaMPs are widely used protein calcium sensors, which are comprised of a fluorescent protein, e.g. GFP, the calcium-binding protein calmodulin (CaM), and CaM-interacting M13 peptide, although a variety of other sensors are also available. Many different proteins are available, including, for example, those described in Zhao et al. (2011) Science 333:1888-1891; Mank et al. (2008) Nat. Methods 5(9):805-11; Akerboom et al. (2012) J. Neurosci. 32(40):13819-40; Chen et al. (2013) Nature 499(7458):295-300; etc.; and as described in U.S. Pat. Nos. 8,629,256, 9,518,980 and 9,488,642 and 9,945,844.

Optogenetics integrates optics and genetic engineering to measure and manipulate neurons. Actuators are genetically-encoded tools for light-activated control of proteins; e.g., opsins and optical switches. Opsins are light-gated ion channels or pumps that absorb light at a specific wavelength. Opsins can be targeted and expressed in specific subsets of neurons, allowing precise spatiotemporal control of these neurons by turning on and off the light source. Channelrhodopsins typically allow the fast depolarization of neurons upon exposure to light through direct stimulation of ion channels. Chlamydomonas reinhardtii Channelrhodopsin-1 (ChR1) is excited by blue light and permits nonspecific cation influx into the cell when stimulated. Examples of ChRs from other species include: CsChR (from Chloromonas subdivisa), CoChR (from Chloromonas oogama), and SdChR (from Scherffelia dubia). Synthetic variants have been created, for example ChR2(H134R), C1V1(t/t), ChIEF; ChETA, VChR1, Chrimson, ChrimsonR, Chronos, PsChR2, CoChR, CsChR, CheRiff, and the like. Alternatively, ChR variants that inhibit neurons have been created and identified, for example GtACR1 and GtACR2 (from the cryptophyte Guillardia theta), and variants such as iChloC, SwiChRca, Phobos, Aurora. Halorhodopsin, known as NpHR (from Natronomonas pharaoni), causes hyperpolarization of the cell when triggered with yellow light, variants include Halo, eNpHR, eNpHR2.0, eNpHR3.0, Jaws. Archaerhodopsin-3 (Arch) from Halorubrum sodomense is also used to inhibit neurons.

The terms “treatment”, “treating”, “treat” and the like are used herein to generally refer to obtaining a desired pharmacologic and/or physiologic effect. The effect may be prophylactic in terms of completely or partially preventing a disease or symptom thereof and/or may be therapeutic in terms of a partial or complete stabilization or cure for a disease and/or adverse effect attributable to the disease. “Treatment” as used herein covers any treatment of a disease in a mammal, particularly a human, and includes: (a) preventing the disease or symptom from occurring in a subject which may be predisposed to the disease or symptom but has not yet been diagnosed as having it; (b) inhibiting the disease symptom, i.e., arresting its development; or (c) relieving the disease symptom, i.e., causing regression of the disease or symptom.

The terms “individual,” “subject,” “host,” and “patient,” are used interchangeably herein and refer to any mammalian subject for whom diagnosis, treatment, or therapy is desired, particularly humans.

### Methods for Producing Spheroids and Assembloids

Methods are provided for the obtention and use of in vitro cell cultures of spheroids (also known as brain region-specific organoids) and assembloids where the spheroids and assembloids are produced from human pluripotent stem cells. Generation of human raphe nuclei spheroids (hRNS) utilizes a multi-step process. Various differentiated spheroid structures, such as hRNS and hCS, are differentiated from spheroids of neural progenitor cells. In some embodiment the human pluripotent stem cells are induced human pluripotent stem (hiPS) cells. In some embodiments the hiPS cells are derived from somatic cells obtained from unaffected individuals. In other embodiments the hiPS cells are derived from somatic cells obtained from an individual comprising at least one allele encoding a mutation associated with a disease, including without limitation the neurologic or psychiatric disorder described above.

Human neural progenitor spheroids. The neural progenitor spheroids can be differentiated from pluripotent stem cells, including without limitation, human induced pluripotent stem cells, hiPS cells. Initially, hiPS cells can be obtained from any convenient source, or can be generated from somatic cells using art-recognized methods. The hiPS cells are dissociated from feeders into single cells and grown in suspension culture, preferably when dissociated as intact colonies. In certain embodiments the culture are feeder layer free, e.g. when grown on vitronectin coated vessels. The culture may further be free on non-human components, i.e. xeno-free. The hiPS cells may be cultured in any medium suitable for the growth and expansion of hiPS cells. For example, the medium may be Essential 8 medium. Suspension growth optionally includes in the culture medium an effective dose of a selective Rho-associated kinase (ROCK) inhibitor for the initial period of culture, for up to about 6 hours, about 12 hours, about 18 hours, about 24 hours, about 36 hours, about 48 hours, (see, for example, Watanabe et al. (2007) Nature Biotechnology 25:681 686). Inhibitors useful for such purpose include, without limitation, Y-27632; Thiazovivin (Cell Res, 2013, 23(10):1187-200; Fasudil (HA-1077) HCl (J Clin Invest, 2014, 124(9):3757-66); GSK429286A (Proc Natl Acad Sci USA, 2014, 111(12):E1140-8); RKI-1447; AT13148; etc. In particular embodiments the ROCK inhibitor Y-27632 is used.

The suspension culture of hiPS cells is then induced to a neural fate. This culture may be feeder-free. For neural induction, an effective dose of an inhibitor of BMP, and of TGFβ pathways is added to the medium (e.g. Essential 8 medium), for a period at least about 2 days, at least about 3 days, at least about 4 days, at least about 5 days, and up to about 10 days, up to about 9 days, up to about 8 days, up to about 7 days, up to about 6 days, up to about 5 days. Such inhibitors are also termed inhibitors of the SMAD pathway. For example, dorsomorphin (DM) can be added at an effective dose of at least about 0.1 μM, at least about 1 JIM, at least about 5 μM, at least about 10 μM, at least about 50 μM, up to about 100 μM concentration, which inhibits bone morphogenetic protein (BMP) type I receptors (ALK2, ALK3 and ALK6). Other useful BMP inhibitors include, without limitation, A 83-01; DMH-1; K 02288; ML 347; SB 505124; etc. SB-431542 is an inhibitor of TGFβ, and can be added at an effective dose of at least about 0.1 μM, at least about 1 μM, at least about 5 μM, at least about 10 μM, at least about 50 μM, up to about 100 μM concentration, which inhibits TGFβ signaling but has no effect on BMP signaling. Other useful inhibitors of TGFβ include, without limitation, LDN-193189 (J Clin Invest, 2015, 125(2):796-808); Galunisertib (LY2157299) (Cancer Res, 2014, 74(21):5963-77); LY2109761 (Toxicology, 2014, 326C:9-17); SB525334 (Cell Signal, 2014, 26(12):3027-35); SD-208; EW-7197; Kartogenin; DMH1; LDN-212854; ML347; LDN-193189 HCl (Proc Natl Acad Sci USA, 2013, 110(52):E5039-48); SB505124; Pirfenidone (Histochem Cell Biol, 2014, 10.1007/s00418-014-1223-0); RepSox; K02288; Hesperetin; GW788388; LY364947, etc. The medium containing the TGFβ and BMP inhibitors may be changed every day.

An effective dose of an inhibitor of GSK-3 may be included in the culture medium. For example, CHIR99021 can be added at an effective dose of from about 0.5 μM to about 50 μM, about 1 μM to about 25 μM, about 1 μM to about 10 μM, about 1 μM to about 5 μM, about 1 μM to about 3 μM, or may be about 1.5 μM. Other useful inhibitors of GSK-3 include, without limitation, CT98014, CT98023, CT99021, TWS119, SB-216763, SB-41528, AR-A014418, AZD-1080 6-BIO, Dibromocantharelline, Hymenialdesine, Indirubin, Meridianin, Alsterpaullone, Cazpaullone, Kenpaullone, etc. The inhibitor of GSK-3 may be added to the medium at the same time as the inhibitor of BMP and inhibitor of TGFβ, or may be added to the medium after about 1, 2, or 3 days following addition of the inhibitor of BMP and inhibitor of TGFβ. For example, the medium may be supplemented with an inhibitor of GSK-3 after 1 to 2 days, e.g. after 1 day, of culture with the inhibitor of BMP and inhibitor of TGFβ.

The method may comprise culturing in the medium comprising the inhibitor of BMP, inhibitor of TGFβ and inhibitor of GSK-3 for a period at least about 2 days, at least about 3 days, at least about 4 days, at least about 5 days, and up to about 10 days, up to about 9 days, up to about 8 days, up to about 7 days, up to about 6 days, up to about 5 days. For example, the neural induction step may comprise culturing in a medium comprising an inhibitor of BMP and an inhibitor of transforming growth factor β TGFβ for a period of 1 to 2 days, e.g. 1 day, supplementing the medium with an inhibitor of GSK-3, and culturing in a medium comprising the inhibitors of BMP, TGFβ and inhibitor of GSK-3 for a period of 4 to 10 days, e.g. 7 days. The medium containing the TGFβ, BMP and GSK-3 inhibitors may be changed every day.

The concentration of inhibitor of BMP may be reduced during culture in the medium. For example, culturing in the medium comprising the inhibitor of BMP, inhibitor of TGFβ and inhibitor of GSK-3 can comprise (1) culturing in a medium comprising the inhibitor of BMP, the inhibitor of TGFβ, and inhibitor of GSK-3 for a period of 2 to 5 days, wherein the inhibitor of TGFβ is present at a concentration of between about 5 μM to about 20 μM, between about 5 μM to about 15 μM between about 8 μM to about 12 μM, or about 10 μM; and subsequently (2) culturing in a medium comprising the inhibitor of BMP, the inhibitor of TGFβ, and inhibitor of GSK-3 for a period of 2 to 5 days, wherein the inhibitor of TGFβ is present at a concentration of between about 1 μM to about 5 μM; about 2 μM to about 4 μM; or about 2.5 μM.

Human raphe nuclei spheroids. After about 5 days, about 6 days, about 7 days, about 8 days, about 9 days, about 10 days in suspension culture, the floating neural progenitor spheroids are moved to neural medium to differentiate the neural progenitors. An exemplary neural medium is a medium comprising neurobasal-medium, B-27 supplement minus vitamin A and a GlutaMAX supplement. The neural medium is supplemented with an inhibitor of GSK-3, a sonic hedgehog pathway agonist, and FGF4.

The inhibitor of GSK-3 may be as set out above. In certain embodiments, the neural medium is supplemented with CHIR99021, for example at a concentration of from about 0.5 μM to about 50 μM, about 1 μM to about 25 μM, about 1 μM to about 10 μM, about 1 μM to about 5 μM, about 1 μM to about 3 μM, or may be about 1.5 μM.

Suitable sonic hedgehog pathway agonists include smoothened agonist, SAG, CAS 364590-63-6, which modulates the coupling of Smo with its downstream effector by interacting with the Smo heptahelical domain (K_{D}=59 nM). SAG may be provided in the neural medium at a concentration of from about 10 nM to about 1 μM, from about 50 nM to about 0.5 μM, from about 75 nM to about 0.25 μM, or may be about 100 nM.

In certain embodiments, the neural medium is supplemented with FGF4, for example at a concentration from about 1 ng/ml to about 100 ng/ml, from about 5 ng/ml to about 50 ng/ml, from about 5 ng/ml to about 15 ng/ml, or about 10 ng/ml. The FGF4 may be added to the neural medium at the same time as the inhibitor of GSK-3 and sonic hedgehog pathway agonist or may be added to the neural medium after at least about 2 days, at least about 3 days, and up to about 10 days, up to about 7 days, up to about 6 days, or up to about 5 days following addition of the inhibitor of BMP and inhibitor of TGFβ. For example, the neural medium may be supplemented with an FGF4 after 1 to 5 days, e.g. after 3 days, of culture in the neural medium with the inhibitor of GSK-3 and sonic hedgehog pathway agonist.

The step of differentiating the neural spheroid into a hRNS may comprise culturing in the neural medium comprising the inhibitor of GSK-3 and sonic hedgehog pathway agonist for a period at least about 2 days, at least about 3 days, and up to about 10 days, up to about 7 days, up to about 6 days, or up to about 5 days. For example, the step of differentiating the neural spheroid into a hRNS may comprise culturing in a neural medium comprising the inhibitor of GSK-3 and sonic hedgehog pathway agonist for a period of 2 to 5 days, e.g. 3 days, supplementing the neural medium with FGF4, and culturing in a neural medium comprising the inhibitor of GSK-3, sonic hedgehog agonist and FGF4 for a period of at least 1 week, at least 2 weeks, at least 3 weeks, up to about 5 weeks, up to about 4 weeks, or between 1 to 3 weeks. The medium containing the inhibitor of GSK-3, sonic hedgehog agonist and FGF4 may be changed every day.

As demonstrated in the examples, the combined use of an inhibitor of GSK-3, a sonic hedgehog pathway agonist, and FGF4 results in the formation of hRNS with high levels of markers indicative of the human raphe nuclei, e.g. at least 2 weeks after the suspension culture of hiPS cells was induced to a neural fate. For example, the hRNS may have high levels of transcription factors that drive caudal midbrain/hindbrain development such as NKX6-1, NKX2-2, OLIG2, GATA2, GATA3, LMX1B, FOXA2, EN1 but low levels of forebrain markers such as FOXG1. Methods for determining levels of transcription factor expression include RT-qPCR as further described in examples. In some embodiments, the methods disclosed herein further comprise determining whether the hRNS express transcription factors that drive caudal midbrain/hindbrain development. A hRNS having high or low levels of a transcription factor may have a significantly higher or lower level of gene expression when compared to gene expression in a non-raphe nuclei spheroid, e.g. a cortical spheroid (hCS), when calculated using a standard statistical test.

To promote differentiation of neural progenitors into neurons, about 1 week, about 2 weeks, about 3 weeks, about 4 weeks after transferring the neural spheroids to the neural medium, the neural medium is changed to replace the inhibitor of GSK-3, a sonic hedgehog pathway agonist with an effective dose of BDNF and NT3. The growth factors can be provided at a concentration for each of at least about 0.5 ng/ml, at least about 1 ng/ml, at least about 5 ng/ml, at least about 10 ng/ml, up to about 500 ng/ml, up to about 250 ng/ml, up to about 100 ng/ml, up to about 20 ng/ml, or about 10 ng/ml.

The neural medium at this stage may be optionally supplemented with an effective dose of one or more of the following agents that promote neuronal activity in general: a gamma secretase inhibitor, e.g. DAPT at a concentration of from about 1 to 25 mM, about 2 to 10 mM, and may be around about 2.5 mM; L-ascorbic acid at a concentration of from about 10 to 500 nM, from about 50 to 250 nM, and may be about 200 nM; cAMP at a concentration of from about 10 to 500 nM, from about 50 to 150 nM, and may be about 100 nM; and Docosahexaenoic acid (DHA) at a concentration of from about 1 μM to 100 μM, from about 5 μM to about 50 μM, from 5 μM to 25 μM, or may be about 10 μM. In some embodiments, the neural medium comprises an effective dose of BDNF, NT3, a gamma secretase inhibitor, L-ascorbic acid, cAMP and DHA.

To promote differentiation of neural progenitors into neurons, the neural spheroids may be cultured in the neural medium comprising the factors listed above for at least about 1 week, at least about 2 weeks, at least about 3 weeks, up to about 6 weeks, up to about 5 weeks, up about 4 weeks, between about 1 and about 3 weeks, or about 2 weeks. The neural medium may further comprise FGF4 for at least about 1 day, at least about 2 days, at least about 3 days, at least about 4 days, at least about 5 days, up to about 10 days, up to about 8 days, up to about 6 days, between about 2 to about 10 days, or about 5 days.

For example, the step of promoting differentiation of neural progenitors into neurons may comprise: (4) culturing the neural spheroid in suspension culture for a period of 2 to 10 days in neural medium comprising FGF4 and at least one of the compounds selected from the group consisting of: brain-derived neurotrophic factor (BDNF), NT-3, L-Ascorbic Acid 2-phosphate Trisodium Salt (AA), N6, 2′-O-Dibutyryladenosine 3′, 5′-cyclic monophosphate sodium salt (cAMP), cis-4, 7, 10, 13, 16, 19-Docosahexaenoic acid (DHA), and DAPT; and (5) culturing the neural spheroid in suspension culture for at least 1 week in neural medium comprising the at least one compound in the absence of FGF4.

About 1 week, 2 weeks, 4 weeks, about 5 weeks, about 6 weeks, about 7 weeks after transferring the neural spheroids to the neural medium, the spheroids can be maintained for extended periods of time in neural medium, e.g. for periods of 1 week, 2 weeks, 3 weeks, 4 weeks, 1, 2, 3, 4, 5, 6, 7, 8, 9, 10, 11, 12 months or longer. In some embodiments, the spheroids are maintained for a period of 3 months or longer. The spheroids may be maintained in a neural medium in the absence of growth factors.

The hRNS comprises functional serotonergic neurons. As mentioned above, a large proportion of neurons that originate in the human raphe nuclei are serotonergic neurons which project into multiple locations of the human central nervous system, including areas of the human cortex. The presence of serotonergic neurons can be detected for example by using methods such as immunohistochemistry to determine expression of serotonin, enzymes involved in the serotonin pathway, such as tryptophan 5-hydroxylase 2 (TPH2), and/or markers of mature serotonin neurons such as vesicular monoamine transporter 2 (VMAT2) and serotonin reuptake transporter (SERT). The functionality of the neurons can be determined by monitoring neuronal activity, e.g. by imaging Ca²⁺ activity.

Human cortical spheroids. hCS may be generated by the methods previously described, for example in Pasca et al. (2015) Nat. Methods 12(7):671-678, entitled “Functional cortical neurons and astrocytes from human pluripotent stem cells in 3D culture”, herein specifically incorporated by reference.

For example, a suspension culture of hiPS cells is cultured to provide a neural progenitor spheroid, as described above. After about 5 days, about 6 days, about 7 days, about 8 days, about 9 days, about 10 days in suspension culture, the floating neural progenitor spheroids are moved to neural media to differentiate the neural progenitors. The media is supplemented with an effective dose of FGF2 and EGF. The growth factors can be provided at a concentration for each of at least about 0.5 ng/ml, at least about 1 ng/ml, at least about 5 ng/ml, at least about 10 ng/ml, at least about 20 ng/ml, up to about 500 ng/ml, up to about 250 ng/ml, up to about 100 ng/ml.

To promote differentiation of neural progenitors into hCS, comprising glutamatergic neurons, after about 1 week, about 2 weeks, about 3 weeks, about 4 weeks after FGF2/EGF exposure the neural medium is changed to replace the FGF2 and EGF with an effective dose of BDNF and NT3. The growth factors can be provided at a concentration for each of at least about 0.5 ng/ml, at least about 1 ng/ml, at least about 5 ng/ml, at least about 10 ng/ml, at least about 20 ng/ml, up to about 500 ng/ml, up to about 250 ng/ml, up to about 100 ng/ml. The cortical spheroids comprise functional glutamatergic neurons.

Assembloids. The hRNS can be functionally integrated with separately cultured human cortical spheroids (hCS), to form cortico-raphe nuclei assembloids (hCS-hRNS) which include glutamatergic and serotoninergic neurons. The resulting hCS-hRNS contains neural circuits between the cortex and raphe nuclei and provides for functional integration of these circuits. For example, the functionally integrated cells interact in a physiologically relevant manner, e.g. forming synapses, transmitting signals, forming multicellular structures, and the like.

The cortical spheroids are co-cultured with the human raphe nuclei spheroids in neural medium under conditions permissive for cell fusion. Condition permissive for cell fusion may include culturing the hRNS and hCS in close proximity, e.g. in direct contact with one another.

Assembly may be performed with spheroids after around about 30 days, about 60 days, about 90 days of culture for hRNS; and after around about 30 days, about 60 days, about 90 days of culture for hCS. The hRNS and hCS spheroids may be co-cultured for a period of 2 days, 3 days, 5 days, 8 days, 10 days, 14 days, 18 days, 21 days or more. Assembly may be carried out in neural medium. The resulting cortico-raphe nuclei assembloids are demonstrated to contain functional neural circuits, where the assembloids comprise bidirectional bidirectional projections between cortical and raphe nuclei spheroids and the serotonergic neurons of the raphe nuclei spheroids were able to modulate activity of cortical neural circuits. Methods for confirming the functionality of the neurons are known in the art and include optogenetic methods and imaging of calcium activity in neurons, such as those methods described in the examples. In some embodiments, the methods may comprise confirming the functionality of the neurons in the cortico-raphe nuclei assembloid.

### Screening Assays

Also disclosed herein are screening assays which involve determining the effect of a candidate agent on the spheroid, e.g. hRNS, or assembloid, e.g. hCS-hRNS, or a cell derived therefrom. A candidate agent may be a small molecule or a genetic agent. The screening assays may involve contacting the candidate agent with the spheroid, assembloid or cell derived therefrom and determining effect of the candidate agent on a parameter of the spheroid, assembloid or cell, where such parameters include morphologic, genetic or functional changes.

For example, a screening assay may involve determining the effect that a candidate agent (e.g. a SSRI inhibitor) has on the functionality of neural circuits within a spheroid or assembloid. As described herein, hCS-hRNS assembloids were demonstrated to comprise neurons with bidirectional projections between the hCS-hRNS and the serotonergic neurons of hRNS were able to modulate function of the cortical neural circuits, as revealed by a combination of viral labeling and calcium imaging with photo-stimulation. The screening assays may therefore involve determining whether a candidate agent is able to alter the ability of the serotonergic neurons to modulate function of the cortical neural circuits in the hCS.

As also described herein, various diseases and disorders are associated with serotonergic dysfunction. Accordingly, the assays described herein may find particular utility where the spheroid or assembloid comprise at least one allele associated with a neurologic or psychiatric disorder, schizophrenia, affective disorder (e.g. MDD, bipolar disorder or an anxiety disorder) and autism spectrum disorder (ASD). Candidate agents that are able to e.g. restore the functionality of neural circuits (e.g. cortical neural circuits) in spheroids or assembloids comprising these disorder-associated alleles may have therapeutic utility in the treatment of said disorder.

Furthermore, the assembloids described herein can be used to dissect cell autonomous contributions in these disorders. For example, an assembloid can be generated where one spheroid (e.g. hRNS or hCS) is derived from a patient suffering from a disorder described herein and the other spheroid is derived from an unaffected individual, i.e. a subject not suffering from the same disorder. For example, in the methods of generating assembloids from a first and second human pluripotent stem cell, either the first or second human pluripotent stem cell can comprise at least one allele associated with a neurologic or psychiatric disorder.

Neural activity causes rapid changes in intracellular free calcium. Calcium imaging assays that exploit this can therefore be used to determine the functional of neuronal circuits. This may involve modifying neurons to contain genetically-encoded calcium indicator proteins, such those proteins that include the fluorophore sensor GCaMP and imaging those cells. GCaMP comprises a circularly permuted green fluorescent protein, a calcium-binding protein calmodulin (CaM) and CaM-interacting M13 peptide, where brightness of the GFP increases upon calcium binding. Further details about calcium imaging assays are described in Chen et al. (2013) Nature 499(7458): 295-300. Other calcium imaging assays include Fura-2 calcium imaging; Fluo-4 calcium imaging, and Cal-590 calcium imaging.

For example, the neurons may be modified to express GCamP6f. This can be combined with methods that activate certain neurons in response to an external stimulus, for example optogenetic methods that activate neurons in response to light. For example, to test functionality between two types of neurons involved in a neural circuit, a “first” neuron can be modified to express an optogenetic actuator (e.g. ChrimsonR) and a “second” neuron modified to express a calcium indicator (e.g. GCamP6f) and imaging used to monitor calcium release. If the first neuron is functionally connected (synapses with) the second neuron, then optogenetic activation of the first neuron will affect intracellular calcium levels and a visible readout in the second neuron.

As described above, serotonin can result in various different responses, which may depend on the receptor serotonin is acting on. Thus, a method of determining the ability of serotonergic neurons to modulate cortical neural circuits may comprise labelling cells of the hRNS with an optogenetic actuator (e.g. ChrimsonR) and labelling cells of the hCS with a calcium indicator, stimulating cells of the hRNS in the hRNS-hCS assembloid and determining whether there is an increase or decrease calcium activity in cells of the hCS in the assembloid. Such increase or decrease may be transient or may occur during the entire post-stimulation period. As set out in the examples herein, such a method was used to confirm serotonergic modulation of the cortical neural circuits in the hRNS-hCS assembloid. To determine the effect a candidate agent has on this serotonergic modulation, this optogenetic method can be carried out in the presence and absence of a candidate agent and the results in the two conditions compared.

Methods of analysis at the single cell level are also of interest, e.g. as described above: live imaging (including confocal or light-sheet microscopy), single cell gene expression or single cell RNA sequencing, calcium imaging, immunocytochemistry, patch-clamping, flow cytometry and the like. Various parameters can be measured to determine the effect of a drug or treatment on the spheroids, assembloids or cells derived therefrom. For example, single cell RNA sequencing of the cells that make up the spheroid or assembloid can be used to characterize the identity of these cells and can be utilized in assays that aim to determine whether a candidate agent affects cell fate.

Parameters are quantifiable components of cells, particularly components that can be accurately measured, desirably in a high-throughput system. A parameter can also be any cell component or cell product including cell surface determinant, receptor, protein or conformational or posttranslational modification thereof, lipid, carbohydrate, organic or inorganic molecule, nucleic acid, e.g. mRNA, DNA, etc. or a portion derived from such a cell component or combinations thereof. While most parameters will provide a quantitative readout, in some instances a semi-quantitative or qualitative result will be acceptable. Readouts may include a single determined value, or may include mean, median value or the variance, etc. Variability is expected and a range of values for each of the set of test parameters will be obtained using standard statistical methods with a common statistical method used to provide single values.

Parameters of interest include detection of cytoplasmic, cell surface or secreted biomolecules, biopolymers, e.g. polypeptides, polysaccharides, polynucleotides, lipids, etc. Cell surface and secreted molecules are a preferred parameter type as these mediate cell communication and cell effector responses and can be more readily assayed. In one embodiment, parameters include specific epitopes. Epitopes are frequently identified using specific monoclonal antibodies or receptor probes. In some cases, the molecular entities comprising the epitope are from two or more substances and comprise a defined structure; examples include combinatorically determined epitopes associated with heterodimeric integrins. A parameter may be detection of a specifically modified protein or oligosaccharide. A parameter may be defined by a specific monoclonal antibody or a ligand or receptor binding determinant.

Candidate agents of interest are biologically active agents that encompass numerous chemical classes, primarily organic molecules, which may include organometallic molecules, inorganic molecules, genetic sequences, etc. An important aspect of the invention is to evaluate candidate drugs, select therapeutic antibodies and protein-based therapeutics, with preferred biological response functions. Candidate agents comprise functional groups necessary for structural interaction with proteins, particularly hydrogen bonding, and typically include at least an amine, carbonyl, hydroxyl or carboxyl group, frequently at least two of the functional chemical groups. The candidate agents often comprise cyclical carbon or heterocyclic structures and/or aromatic or polyaromatic structures substituted with one or more of the above functional groups. Candidate agents are also found among biomolecules, including peptides, polynucleotides, saccharides, fatty acids, steroids, purines, pyrimidines, derivatives, structural analogs or combinations thereof.

Also included are pharmacologically active drugs, genetically active molecules, etc. Compounds of interest include chemotherapeutic agents, anti-inflammatory agents, hormones or hormone antagonists, ion channel modifiers, and neuroactive agents. Exemplary of pharmaceutical agents suitable for this invention are those described in, “The Pharmacological Basis of Therapeutics,” Goodman and Gilman, McGraw-Hill, New York, N.Y., (1996), Ninth edition, under the sections: Drugs Acting at Synaptic and Neuroeffector Junctional Sites; Cardiovascular Drugs; Vitamins, Dermatology; and Toxicology, all incorporated herein by reference.

An important class of candidate agents for use with the compositions and methods described herein are selective serotonin reuptake inhibitors (SSRIs). SSRIs are a class of drug that are typically used as antidepressants in the treatment of major depressive disorder (MDD) and anxiety disorders. SSRIs typically function by increasing the extracellular level of serotonin by limiting its reabsorption. Examples of known SSRIs include Citalopram, Escitalopram, Fluoxetine, Fluvoxamine, Paroxetine, Sertraline, Dapoxetine. As well as investigating the role of these known SSRIs, the systems described here can also be used as part of a screening assay to discover new SSRIs.

Test compounds include all of the classes of molecules described above, and may further comprise samples of unknown content. Of interest are complex mixtures of naturally occurring compounds derived from natural sources such as plants. While many samples will comprise compounds in solution, solid samples that can be dissolved in a suitable solvent may also be assayed. Samples of interest include environmental samples, e.g. ground water, sea water, mining waste, etc.; biological samples, e.g. lysates prepared from crops, tissue samples, etc.; manufacturing samples, e.g. time course during preparation of pharmaceuticals; as well as libraries of compounds prepared for analysis; and the like. Samples of interest include compounds being assessed for potential therapeutic value, i.e. drug candidates.

The term samples also include the fluids described above to which additional components have been added, for example components that affect the ionic strength, pH, total protein concentration, etc. In addition, the samples may be treated to achieve at least partial fractionation or concentration. Biological samples may be stored if care is taken to reduce degradation of the compound, e.g. under nitrogen, frozen, or a combination thereof. The volume of sample used is sufficient to allow for measurable detection, usually from about 0.1 to 1 ml of a biological sample is sufficient.

Compounds, including candidate agents, are obtained from a wide variety of sources including libraries of synthetic or natural compounds. For example, numerous means are available for random and directed synthesis of a wide variety of organic compounds, including biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

As used herein, the term “genetic agent” refers to polynucleotides and analogs thereof, which agents are tested in the screening assays of the invention by addition of the genetic agent to a cell. The introduction of the genetic agent results in an alteration of the total genetic composition of the cell. Genetic agents such as DNA can result in an experimentally introduced change in the genome of a cell, generally through the integration of the sequence into a chromosome, for example using CRISPR mediated genomic engineering (see for example Shmakov et al. (2017) Nature Reviews Microbiology 15:169). Genetic changes can also be transient, where the exogenous sequence is not integrated but is maintained as an episomal agents. Genetic agents, such as antisense oligonucleotides, can also affect the expression of proteins without changing the cell's genotype, by interfering with the transcription or translation of mRNA. The effect of a genetic agent is to increase or decrease expression of one or more gene products in the cell.

Introduction of an expression vector encoding a polypeptide can be used to express the encoded product in cells lacking the sequence, or to over-express the product. Various promoters can be used that are constitutive or subject to external regulation, where in the latter situation, one can turn on or off the transcription of a gene. These coding sequences may include full-length cDNA or genomic clones, fragments derived therefrom, or chimeras that combine a naturally occurring sequence with functional or structural domains of other coding sequences. Alternatively, the introduced sequence may encode an anti-sense sequence; be an anti-sense oligonucleotide; RNAi, encode a dominant negative mutation, or dominant or constitutively active mutations of native sequences; altered regulatory sequences, etc. The expression vector may be a viral vector, e.g. adeno-associated virus, adenovirus, herpes simplex virus, retrovirus, lentivirus, alphavirus, flavivirus, rhabdovirus, measles virus, Newcastle disease virus, poxvirus and picornavirus vectors.

Antisense and RNAi oligonucleotides can be chemically synthesized by methods known in the art. Preferred oligonucleotides are chemically modified from the native phosphodiester structure, in order to increase their intracellular stability and binding affinity. A number of such modifications have been described in the literature, which alter the chemistry of the backbone, sugars or heterocyclic bases. Among useful changes in the backbone chemistry are phosphorothioates; phosphorodithioates, where both of the non-bridging oxygens are substituted with sulfur; phosphoroamidites; alkyl phosphotriesters and boranophosphates. Achiral phosphate derivatives include 3′-O′-5′-S-phosphorothioate, 3′-S-5′-O-phosphorothioate, 3′-CH2-5′-O-phosphonate and 3′-NH-5′-O-phosphoroamidate. Peptide nucleic acids replace the entire ribose phosphodiester backbone with a peptide linkage. Sugar modifications are also used to enhance stability and affinity, e.g. morpholino oligonucleotide analogs.

A plurality of assays may be run in parallel with different agent concentrations to obtain a differential response to the various concentrations. As known in the art, determining the effective concentration of an agent typically uses a range of concentrations resulting from 1:10, or other log scale, dilutions. The concentrations may be further refined with a second series of dilutions, if necessary. Typically, one of these concentrations serves as a negative control, i.e. at zero concentration or below the level of detection of the agent or at or below the concentration of agent that does not give a detectable change in the phenotype.

Various methods can be utilized for quantifying the presence of selected parameters, in addition to the functional parameters described above. For measuring the amount of a molecule that is present, a convenient method is to label a molecule with a detectable moiety, which may be fluorescent, luminescent, radioactive, enzymatically active, etc., particularly a molecule specific for binding to the parameter with high affinity fluorescent moieties are readily available for labeling virtually any biomolecule, structure, or cell type. Immunofluorescent moieties can be directed to bind not only to specific proteins but also specific conformations, cleavage products, or site modifications like phosphorylation. Individual peptides and proteins can be engineered to fluoresce, e.g. by expressing them as green fluorescent protein chimeras inside cells (for a review see Jones et al. (1999) Trends Biotechnol. 17(12):477-81). Thus, antibodies can be genetically modified to provide a fluorescent dye as part of their structure

Depending upon the label chosen, parameters may be measured using other than fluorescent labels, using such immunoassay techniques as radioimmunoassay (RIA) or enzyme linked immunoabsorbance assay (ELISA), homogeneous enzyme immunoassays, and related non-enzymatic techniques. These techniques utilize specific antibodies as reporter molecules, which are particularly useful due to their high degree of specificity for attaching to a single molecular target. U.S. Pat. No. 4,568,649 describes ligand detection systems, which employ scintillation counting. These techniques are particularly useful for protein or modified protein parameters or epitopes, or carbohydrate determinants. Cell readouts for proteins and other cell determinants can be obtained using fluorescent or otherwise tagged reporter molecules. Cell based ELISA or related non-enzymatic or fluorescence-based methods enable measurement of cell surface parameters and secreted parameters. Capture ELISA and related non-enzymatic methods usually employ two specific antibodies or reporter molecules and are useful for measuring parameters in solution. Flow cytometry methods are useful for measuring cell surface and intracellular parameters, as well as shape change and granularity and for analyses of beads used as antibody- or probe-linked reagents. Readouts from such assays may be the mean fluorescence associated with individual fluorescent antibody-detected cell surface molecules or cytokines, or the average fluorescence intensity, the median fluorescence intensity, the variance in fluorescence intensity, or some relationship among these.

Both single cell multiparameter and multicell multiparameter multiplex assays, where input cell types are identified and parameters are read by quantitative imaging and fluorescence and confocal microscopy are used in the art, see Confocal Microscopy Methods and Protocols (Methods in Molecular Biology Vol. 122.) Paddock, Ed., Humana Press, 1998. These methods are described in U.S. Pat. No. 5,989,833 issued Nov. 23, 1999.

The results of an assay can be entered into a data processor to provide a dataset. Algorithms are used for the comparison and analysis of data obtained under different conditions. The effect of factors and agents is read out by determining changes in multiple parameters. The data will include the results from assay combinations with the agent(s), and may also include one or more of the control state, the simulated state, and the results from other assay combinations using other agents or performed under other conditions. For rapid and easy comparisons, the results may be presented visually in a graph, and can include numbers, graphs, color representations, etc.

The dataset is prepared from values obtained by measuring parameters in the presence and absence of different cells, e.g. genetically modified cells, cells cultured in the presence of specific factors or agents that affect neuronal function, as well as comparing the presence of the agent of interest and at least one other state, usually the control state, which may include the state without agent or with a different agent. The parameters include functional states such as synapse formation and calcium ions in response to stimulation, whose levels vary in the presence of the factors. Desirably, the results are normalized against a standard, usually a “control value or state,” to provide a normalized data set. Values obtained from test conditions can be normalized by subtracting the unstimulated control values from the test values, and dividing the corrected test value by the corrected stimulated control value. Other methods of normalization can also be used; and the logarithm or other derivative of measured values or ratio of test to stimulated or other control values may be used. Data is normalized to control data on the same cell type under control conditions, but a dataset may comprise normalized data from one, two or multiple cell types and assay conditions.

The dataset can comprise values of the levels of sets of parameters obtained under different assay combinations. Compilations are developed that provide the values for a sufficient number of alternative assay combinations to allow comparison of values.

A database can be compiled from sets of experiments, for example, a database can contain data obtained from a panel of assay combinations, with multiple different environmental changes, where each change can be a series of related compounds, or compounds representing different classes of molecules.

Mathematical systems can be used to compare datasets, and to provide quantitative measures of similarities and differences between them. For example, the datasets can be analyzed by pattern recognition algorithms or clustering methods (e.g. hierarchical or k-means clustering, etc.) that use statistical analysis (correlation coefficients, etc.) to quantify relatedness. These methods can be modified (by weighting, employing classification strategies, etc.) to optimize the ability of a dataset to discriminate different functional effects. For example, individual parameters can be given more or less weight when analyzing the dataset, in order to enhance the discriminatory ability of the analysis. The effect of altering the weights assigned each parameter is assessed, and an iterative process is used to optimize pathway or cellular function discrimination.

The comparison of a dataset obtained from a test compound, and a reference dataset(s) is accomplished by the use of suitable deduction protocols, AI systems, statistical comparisons, etc. Preferably, the dataset is compared with a database of reference data. Similarity to reference data involving known pathway stimuli or inhibitors can provide an initial indication of the cellular pathways targeted or altered by the test stimulus or agent.

A reference database can be compiled. These databases may include reference data from panels that include known agents or combinations of agents that target specific pathways, as well as references from the analysis of cells treated under environmental conditions in which single or multiple environmental conditions or parameters are removed or specifically altered. Reference data may also be generated from panels containing cells with genetic constructs that selectively target or modulate specific cellular pathways. In this way, a database is developed that can reveal the contributions of individual pathways to a complex response.

The effectiveness of pattern search algorithms in classification can involve the optimization of the number of parameters and assay combinations. The disclosed techniques for selection of parameters provide for computational requirements resulting in physiologically relevant outputs. Moreover, these techniques for pre-filtering data sets (or potential data sets) using cell activity and disease-relevant biological information improve the likelihood that the outputs returned from database searches will be relevant to predicting agent mechanisms and in vivo agent effects.

For the development of an expert system for selection and classification of biologically active drug compounds or other interventions, the following procedures are employed. For every reference and test pattern, typically a data matrix is generated, where each point of the data matrix corresponds to a readout from a parameter, where data for each parameter may come from replicate determinations, e.g. multiple individual cells of the same type. As previously described, a data point may be quantitative, semi-quantitative, or qualitative, depending on the nature of the parameter.

The readout may be a mean, average, median or the variance or other statistically or mathematically derived value associated with the measurement. The parameter readout information may be further refined by direct comparison with the corresponding reference readout. The absolute values obtained for each parameter under identical conditions will display a variability that is inherent in live biological systems and also reflects individual cellular variability as well as the variability inherent between individuals.

Classification rules are constructed from sets of training data (i.e. data matrices) obtained from multiple repeated experiments. Classification rules are selected as correctly identifying repeated reference patterns and successfully distinguishing distinct reference patterns. Classification rule-learning algorithms may include decision tree methods, statistical methods, naive Bayesian algorithms, and the like.

A knowledge database will be of sufficient complexity to permit novel test data to be effectively identified and classified. Several approaches for generating a sufficiently encompassing set of classification patterns, and sufficiently powerful mathematical/statistical methods for discriminating between them can accomplish this.

The data from cells treated with specific drugs known to interact with particular targets or pathways provide a more detailed set of classification readouts. Data generated from cells that are genetically modified using over-expression techniques and anti-sense techniques, permit testing the influence of individual genes on the phenotype.

A preferred knowledge database contains reference data from optimized panels of cells, environments and parameters. For complex environments, data reflecting small variations in the environment may also be included in the knowledge database, e.g. environments where one or more factors or cell types of interest are excluded or included or quantitatively altered in, for example, concentration or time of exposure, etc

For further elaboration of general techniques useful in the practice of this invention, the practitioner can refer to standard textbooks and reviews in cell biology, tissue culture, embryology, and neurobiology. With respect to tissue culture and embryonic stem cells, the reader may wish to refer to Teratocarcinomas and embryonic stem cells: A practical approach (E. J. Robertson, ed., IRL Press Ltd. 1987); Guide to Techniques in Mouse Development (P. M. Wasserman et al. eds., Academic Press 1993); Embryonic Stem Cell Differentiation in Vitro (M. V. Wiles, Meth. Enzymol. 225:900, 1993); Properties and uses of Embryonic Stem Cells: Prospects for Application to Human Biology and Gene Therapy (P. D. Rathjen et al., Reprod. Fertil. Dev. 10:31, 1998).

General methods in molecular and cellular biochemistry can be found in such standard textbooks as Molecular Cloning: A Laboratory Manual, 3rd Ed. (Sambrook et al., Harbor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998). Reagents, cloning vectors, and kits for genetic manipulation referred to in this disclosure are available from commercial vendors such as BioRad, Stratagene, Invitrogen, Sigma-Aldrich, and ClonTech.

Each publication cited in this specification is hereby incorporated by reference in its entirety for all purposes.

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### EXPERIMENTAL

Described herein is a novel approach to study functional neuromodulatory systems using human pluripotent stem cells (hPSCs) to generate three-dimensional (3D) assembloids that contain forebrain organoids coupled to organoids that model the raphe nuclei and that are capable of sending serotonergic projections. We first generated hPSC-derived raphe nuclei spheroids (hRNS), which include functional serotonergic neurons along with other resident cell types of raphe nuclei. Assembly of human cerebral cortical spheroids (hCSs), which include pyramidal glutamatergic neurons of various cortical layers, with hRNS resulted in hRNS-hCS assembloids within which bidirectional projections between cortex and raphe nuclei are present. Using a combination of single-cell RNA sequencing, viral labelling and calcium imaging with photo-stimulation, we demonstrated an in vitro, human cellular model of serotonergic modulation of cortical circuits, which could be used as a platform for understanding the assembly of these circuits, modelling of disorders such as MDD, autism spectrum disorder and schizophrenia, and developing drug screening to identify better drugs targeting this neuromodulatory pathway.

### Example 1

### Methods

Generation of human raphe nuclei spheroids (hRNS). Human pluripotent stem cells (hPSCs) were maintained on six-well plates coated with recombinant human vitronectin (VTN-N, Life Technologies, A14700) in Essential 8 medium (Life Technologies, A1517001) supplemented with penicillin and streptomycin (1:100, Gibco, 15140122). To generate hRNS, hiPSCs were incubated with Accutase (Innovate Cell Technologies, AT-104) at 37° C. for 7 min and dissociated into single cells. Approximately 3 million single cells were added per AggreWell 800 (STEMCELL Technologies, 34815) well in Essential 8 medium supplemented with the ROCK inhibitor Y-27632 (10 μM, Selleckchem, S1049), centrifuged at 100 g for 3 min to capture the cells in the microwells and incubated at 37° C. with 5% CO2. After 24 hours, we collected spheroids from each microwell by firmly pipetting (with a cut end of a P1000 tip) medium in the well up and down and transferring it into ultra-low-attachment plastic dishes (Corning, 3262) in Essential 6 medium (Life Technologies, A1516401) supplemented with dorsomorphin (2.5 μM, Sigma-Aldrich, P5499) and SB-431542 (10 μM, Tocris, 1614). From day 2 to 8, medium was changed every day and supplemented with dorsomorphin, SB-431542 (10 μM from day 2 to 5, 2.5 μM from day 5 to 8) and GSK-3 inhibitor CHIR 99021 (1.5 μM).

On day 6, neural spheroids were transferred to neural medium containing Neurobasal A (Life Technologies, 10888), B-27 supplement without vitamin A (Life Technologies, 12587), GlutaMax (1:100, Life Technologies, 35050) and penicillin and streptomycin. From day 5 to day 15, neural medium was changed every day and was supplemented with CHIR 99021 (1.5 μM) and Smoothened agonist SAG (100 nM). From day 8 to 20, neural medium was also supplemented with fibroblast growth factor-4 (FGF4, 10 ng/mL).

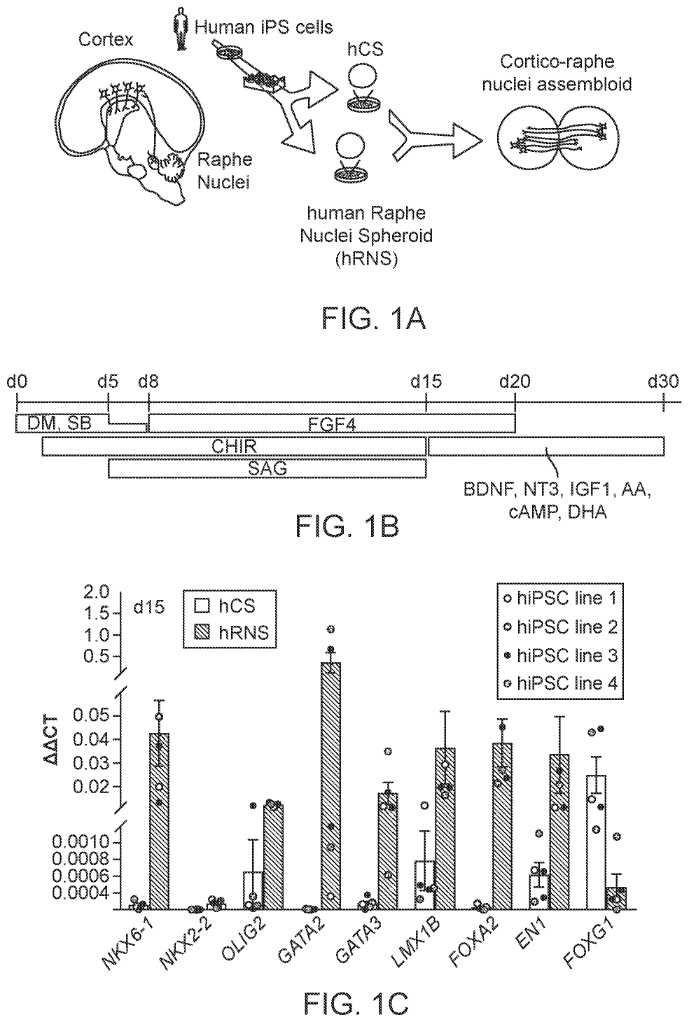

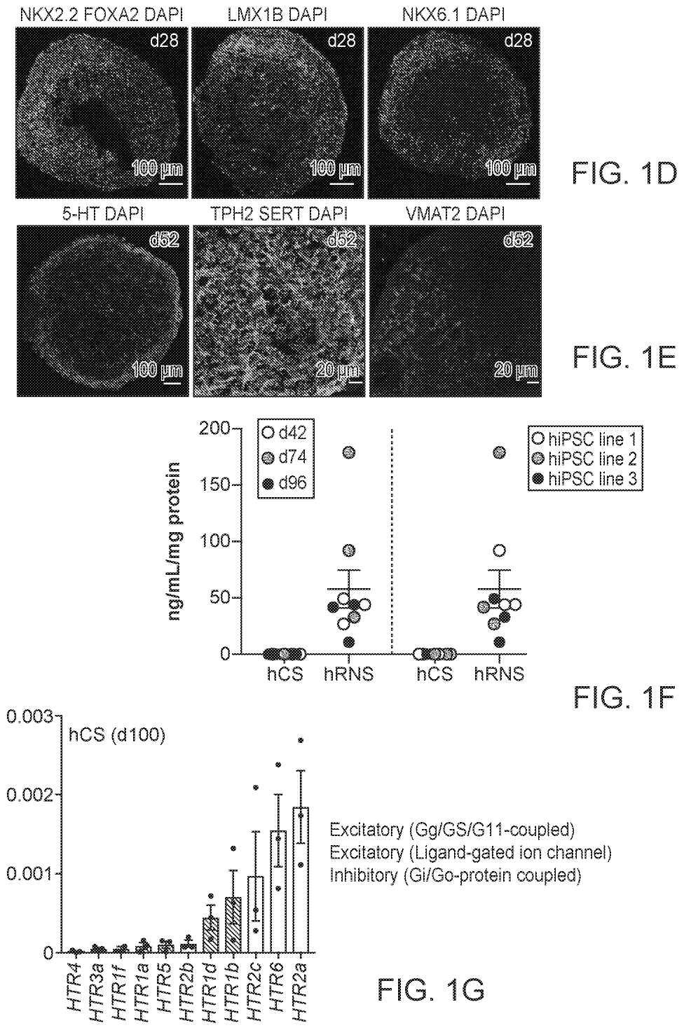

To promote differentiation, from day 16 to 30, neural medium was changed every other day and was supplemented with BDNF (10 ng/mL), NT-3 (10 ng/mL), IGF-1 (10 ng/mL), cAMP (100 nM), L-ascorbic acid (200 μM) and Docosahexaenoic Acid (DHA, 10 μM). A schematic showing the different recipes is presented in FIG. 1A. To characterize cellular diversity in hRNS, we performed single cell transcriptomics on dissociated hRNS at day 42 based of supplier's recommendations (10× genomics, 120262). To quantify serotonin release in hRNS, 2-3 intact hRNS per timepoint per hiPS cell line were flash frozen at various timepoints and processed for high performance liquid chromatography (HPLC). For serotonin uncaging experiments, NPEC caged-serotonin (Tocris, 3991) was used at a final concentration of 50 μM. The FRAP module of the Leica SP8 confocal microscope was used to uncage glutamate using UV light (405 nm).

Generation of cortico-raphe nuclei assembloids (hCS-hRNS). (FIG. 1A) To generate cortico-raphe cortico-raphe nuclei assembloids (hCS-hRNS), hCS and hRNS were generated separately, and later assembled by placing them in close proximity with each other in 1.5 ml microcentrifuge tubes for 3 days in an incubator. Neural media used for assembly contained neurobasal-A, B-27 supplement without vitamin A, GlutaMax (1:100), penicillin and streptomycin (1:100). Media was carefully changed on day 2, and on the 3V day, assembloids were placed in 24-well ultra-low attachment plates in the neural medium described above using a cut P1000 pipette tip. After this, media was changed every 3-4 days. hCS was generated by previously described methods13, 14. Assembly was performed between days 45 and 60. For some experiments hCS or hRNS were virally labeled with AAV-DJ1-hSyn1::YFP seven to ten days prior to assembly. For optogenetic photo-stimulation experiment, hRNS were labeled with AAV1-hSyn1::ChrimsonR-tdTomato virus and assembled with EF1a-GCaMP6s-expressing hCS as described above.

### Example 2

### Generation of Functional hRNS

To specify spheroids (organoids) resembling raphe nuclei, hPSCs aggregated in microwells were first patterned by double SMAD inhibition towards neuroectoderm and later exposed to CHIR, the SHH agonist SAG and FGF4 (FIG. 1B). Gene and protein expression analysis by RT-qPCR and immunocytochemistry at day 15 of patterning showed upregulation of transcription factors that drive caudal midbrain/hindbrain development (NKX6-1, NKX2-2, OLIG2, GATA2, GATA3, LMX1B, FOXA2, EN1; FIG. 1C, D) and downregulation of forebrain marker FOXG1 (FIG. 1C).

Immunocytochemistry at day 52 showed the presence of serotonergic neurons characterized by the presence of 5-hydroxytryptamine (serotonin, 5-HT) and one of the principle enzymes in serotonin synthesis pathway tryptophan 5-hydroxylase 2 (TPH2). The core molecular phenotype of mature serotonergic neurons includes vesicular monoamine transporter 2 (VMAT2), which packages 5-HT into synaptic vesicles and serotonin reuptake transporter SERT and recycles extracellular 5-HT15. Immunocytochemistry at day 52 revealed cells positive for both SERT and VMAT2 in hRNS (FIG. 1E). Next, we used HPLC to measure 5-HT release in hRNS. Across three different timepoints and lines, we consistently measured 5-HT in hRNS ranging from 20-175 ng/mL/mg protein, whereas no detectable 5-HT was present in any of the timepoints for hCS, demonstrating the specificity of hRNS patterning towards raphe nuclei (FIG. 1F). Next, surveyed the gene expression profiles for eleven 5HT metabotropic receptors (HTRs) in hCS. We observed that a combination of excitatory Go/Gs/G11-coupled (in pink, HTR2a, HTR6, HTR2c) and inhibitory Gi/Go-coupled (in green, HTR1b, 1d) were expressed in hCS at day 100 (FIG. 1G).

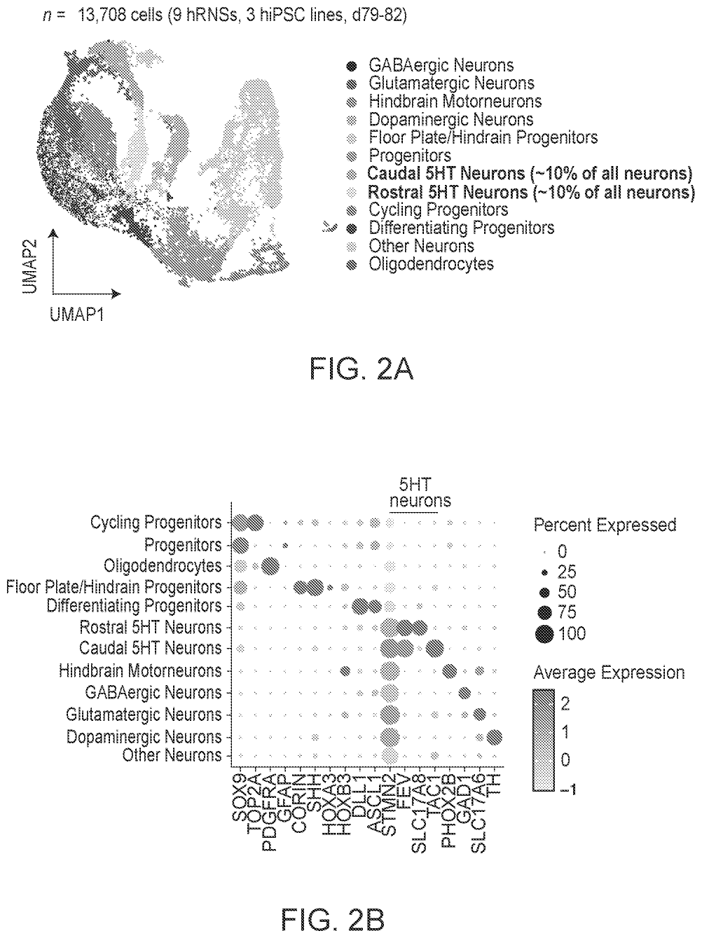

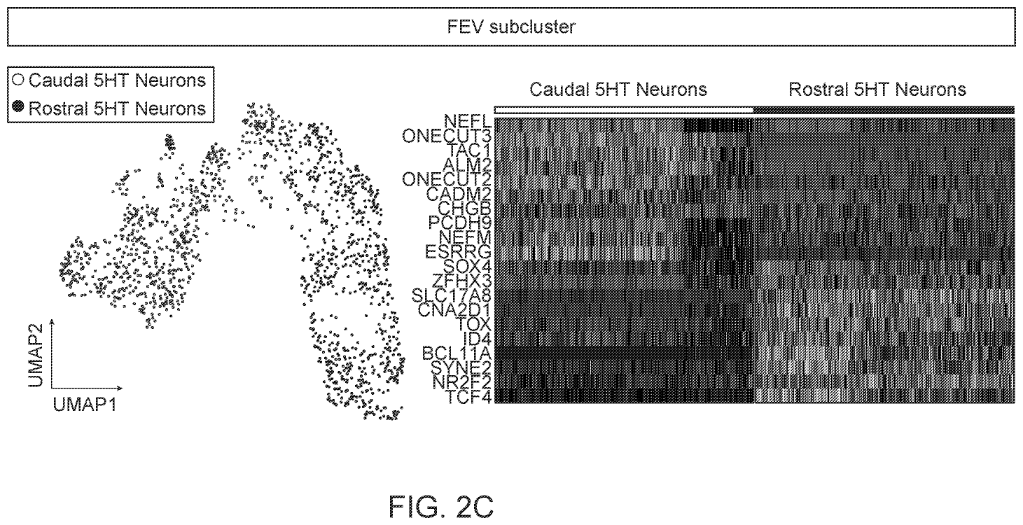

To probe the diversity of cell types in hRNS, we performed single cell transcriptomics of hRNS on day 79-82. Unsupervised clustering of hRNS cells revealed large populations of hindbrain-lineage neurons expressing classical markers for the 5HT-lineage markers (“5HT Neurons”) as well as other neuronal subtypes (“GABAergic Neurons”, “Glutamatergic Neurons”) (FIG. 2A-B). Closer inspection of the 5-HT cluster revealed caudal and rostral subpopulations (FIG. 2C)

### Example 3

### Assembly of hCS-hRNS

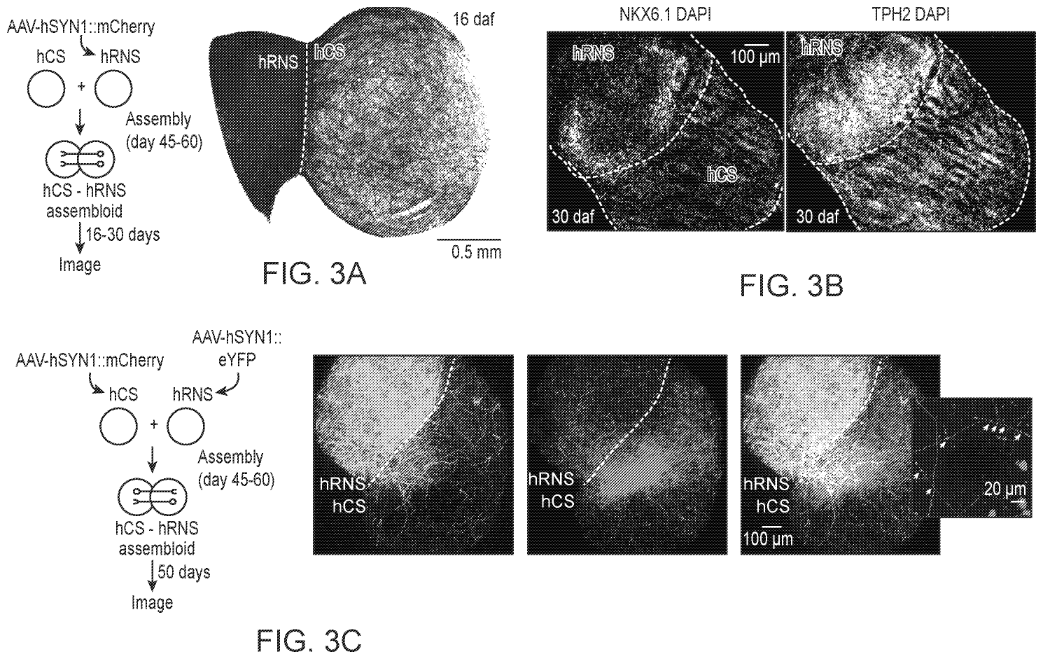

To model the development and function of the cortico-raphe nuclei circuitry, hRNS were virally labeled using AAV-DJ1-hSYN1::mCherry between days 45 and 60 and assembled with hCS 7-8 days later, resulting in hRNS-hCS assembloids. Live imaging of intact hRNS-hCS 16 days after assembly (days after fusion; daf) showed hRNS-derived mCherry⁺ cells extensively projecting to hCS (FIG. 3A). Immunocytochemistry additionally showed TPH2⁺ cells projecting into the hCS (FIG. 3B). To assess the directionality of projections in hRNS-hCS, we virally labelled hRNS and hCS with AAV-DJ1-hSYN1::eYFP and AAV-DJ1-hSYN1::mCherry respectively, and subsequently assembled them. Live imaging of intact hRNS-hCS 50 days after assembly revealed bidirectional projections between hRNS and hCS. Forebrain-projecting serotonergic cells in raphe nuclei display distinct axonal morphologies with large and oval varicosities along thin axons [4]. Similar structures were observed along the axons of hRNS-derived eYFP⁺ projections in hCS, and not in hCS-derived mCherry+ projections (FIG. 3C).

### Example 4

### Functional Probing of Neuromodulatory Connectivity in hCS-hRNS

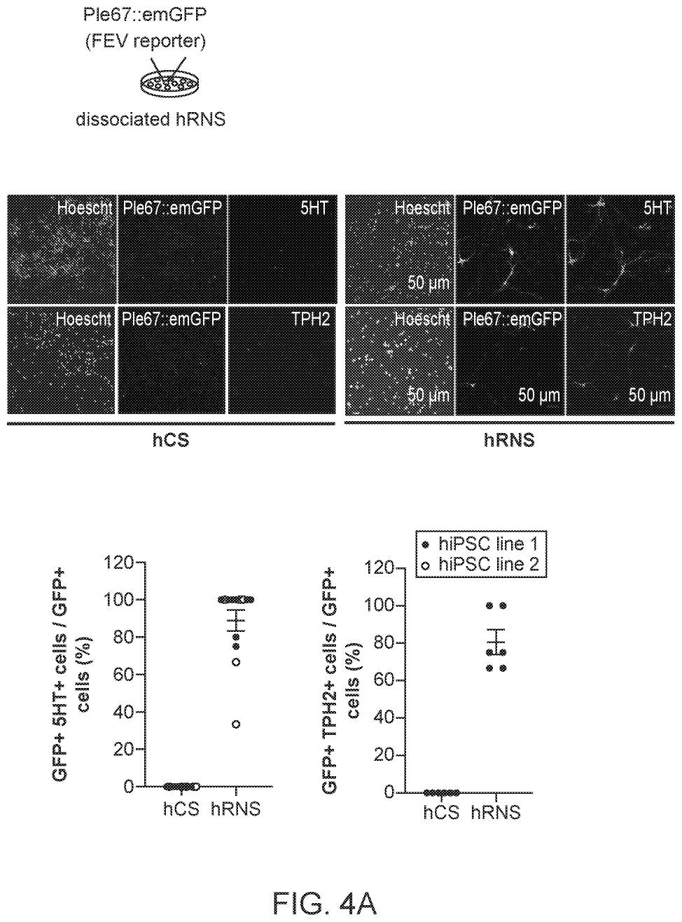

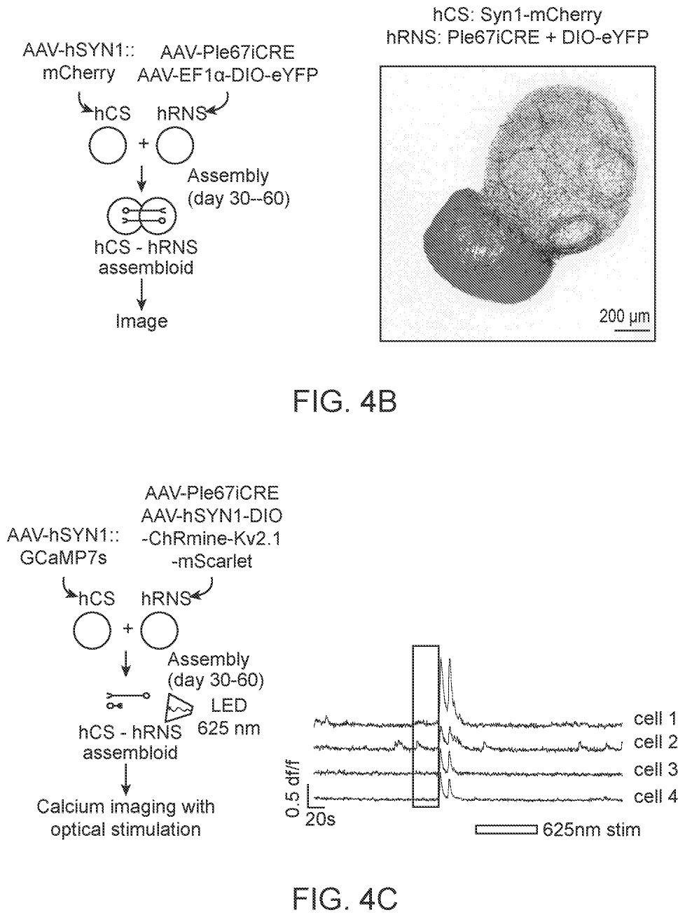

To label the 5HT-lineage neurons in hRNS for functional studies, we used a viral reporter that drives expression of emGFP under the FEV minipromoter Ple67 (AAV-Ple67::emGFP). We characterized its specificity using immunostainings for 5-HT and TPH2 on dissociated hRNS cells infected with Ple67::emGFP (FIG. 4A); this experiments showed that 80-90% of all emGFP⁺ were 5HT⁺ or TPH2⁺ indicating high specificity. Next, we used an iCRE-dependent version of this reporter (AAV-DJ-Ple67iCRE) and co-infected hRNS with AAV-EF1α-DIO-eYFP to induce recombination and drive eYFP expression in 5-HT-lineage cells. We then assembled co-infected hRNS with hCS infected with AAV-hSYN1::mCherry. The resulting hCS-hRNS showed extensive eYFP⁺ projections of 5-HT-lineage cells from hRNS into hCS (FIG. 4B). To functionally probe serotonergic input into the hCS in hCS-hRNS, we used the same iCRE-dependent Ple67 reporter to express ChRmine-K_{V}2.1, which is a soma-targeted red-shifted opsin, in 5HT-lineage cells of hRNS (AAV-Ple67iCRE and AAV-EF1α-DIO-ChRmine-K_{V}2.1) and assembled them with hCS labelled with a genetically encoded calcium indicator (AAV-hSYN1-GCamP7s). Photo-stimulation of 5-HT-lineage cells performed by high-frequency optical stimulations at the 625 nm wavelength reliably evoked responses in the hCS neurons as indicated by the stimulation-locked calcium responses (FIG. 4C).

### CITATIONS

Nadim, F. & Bucher, D. Neuromodulation of Neurons and Synapses. Curr. Opin. Neurobiol. 0, 48-56 (2014). Bucher, D. & Marder, E. SnapShot: Neuromodulation. Cell 155, 482-482.e1 (2013). Vitalis, T. & Pamavelas, J. G. The Role of Serotonin in Early Cortical Development. Dev. Neurosci. 25, 245-256 (2003). Hornung, J.-P. The human raphe nuclei and the serotonergic system. J. Chem. Neuroanat. 26, 331-343 (2003). Jacobs, B. L. & Azmitia, E. C. Structure and function of the brain serotonin system. Physiol. Rev. 72, 165-229 (1992). Hodge, R. D. et al. Conserved cell types with divergent features between human and mouse cortex. bioRxiv384826 (2018). doi:10.1101/384826 Sundström, E. et al. Neurochemical differentiation of human bulbospinal monoaminergic neurons during the first trimester. Dev. Brain Res. 75, 1-12 (1993). Takahash et al. Distribution of serotonin-containing cell bodies in the brainstem of the human fetus determined with immunohistochemistry using antiserotonin serum. Brain Dev. 8, 355-365 (1986). Adell, A. Revisiting the role of raphe and serotonin in neuropsychiatric disorders. J. Gen. Physiol. 145, 257-259 (2015). Sodhi, M. S. K. & Sanders-Bush, E. Serotonin and brain development. in International Review of Neurobiology 59, 111-174 (Academic Press, 2004). Bonnin, A. et al. A transient placental source of serotonin for the fetal forebrain. Nature 472, 347-350 (2011). Whitaker-Azmitia, P. M. Serotonin and brain development: role in human developmental diseases. Brain Res. Bull. 56, 479-485 (2001). Paşca, A. M. et al. Functional cortical neurons and astrocytes from human pluripotent stem cells in 3D culture. Nat. Methods 12, 671-678 (2015). Birey, F. et al. Assembly of functionally integrated human forebrain spheroids. Nature 545, 54-59 (2017). Okaty, B. W., Commons, K. G. & Dymecki, S. M. Embracing diversity in the 5-HT neuronal system. Nat. Rev. Neurosci. 1 (2019). doi:10.1038/s41583-019-0151-3. Celada, P., Puig, M. V. & Artigas, F. Serotonin modulation of cortical neurons and networks. Front. Integr. Neurosci. 7, (2013). Frank, C. Recognition and treatment of serotonin syndrome. Can. Fam. Physician 54, 988-992 (2008). Walsh, J. J. et al. 5-HT release in nucleus accumbens rescues social deficits in mouse autism model. Nature 560, 589 (2018). Doan, R. N. et al. Recessive gene disruptions in autism spectrum disorder. Nat. Genet. 1(2019). doi:10.1038/s41588-019-0433-8.

1. A method for producing a human raphe nuclei-like spheroid or organoid (hRNS) in vitro, the method comprising:
(a) inducing a human pluripotent stem cell in 3D suspension culture to a neural fate to generate a neural spheroid by culturing in a medium comprising an inhibitor of bone morphogenetic protein (BMP) and an inhibitor of transforming growth factor β (TGFβ), and wherein the method comprises supplementing the medium with an inhibitor of GSK-3 for a period of from 2 to 10 days;
(b) differentiating the neural spheroid into a hRNS by culturing the neural spheroid in suspension culture in neural medium comprising an inhibitor of GSK-3 and a sonic hedgehog pathway agonist, and supplementing the neural medium with FGF4; and
(c) maintaining the hRNS in neural medium for at least one week, such that the hRNS comprises serotonergic neurons.

2-3. (canceled)

4. The method of claim 1, wherein the inhibitor of BMP is dorsomorphin and the inhibitor of TGFβ is SB-431542 and the inhibitor of GSK-3 is CHIR99021, optionally wherein the medium is supplemented with the inhibitor of GSK-3 after a period of 1 to 2 days such that the medium comprises the inhibitor of BMP, inhibitor of TGFβ and inhibitor of GSK-3, optionally wherein culturing in the medium comprising the inhibitor of BMP, inhibitor of TGFβ and inhibitor of GSK-3 is for a period of from 4 to 8 days.

5-7. (canceled)

8. The method of claim 1, wherein in step (a) culturing in the medium comprising the inhibitor of BMP, inhibitor of TGFβ and inhibitor of GSK-3 comprises:
(1) culturing in a medium comprising the inhibitor of BMP, the inhibitor of TGFβ, and inhibitor of GSK-3 for a period of 2 to 5 days, wherein the inhibitor of TGFβ is present at a concentration of between 5 μM to 20 μM; and subsequently
(2) culturing in a medium comprising the inhibitor of BMP, the inhibitor of TGFβ, and inhibitor of GSK-3 for a period of 2 to 5 days, wherein the inhibitor of TGFβ is present at a concentration of between 1 μM to 5 μM.

9-12. (canceled)

13. The method of claim 1, wherein in step (b) the neural spheroid is cultured in neural medium comprising the inhibitor of GSK-3 and a SHH pathway agonist for a period of 1 to 3 weeks, and wherein the neural medium is supplemented with FGF4 after a period of 2 to 10 days.

14. The method of claim 1, wherein step (b) further comprises supplementing the neural medium with at least one of the compounds selected from the group consisting of: brain-derived neurotrophic factor (BDNF), NT3, L-Ascorbic Acid 2-phosphate Trisodium Salt (AA), N6, 2′-O-Dibutyryladenosine 3′, 5′-cyclic monophosphate sodium salt (cAMP), cis-4, 7, 10, 13, 16, 19-Docosahexaenoic acid (DHA), and DAPT,
optionally wherein the neural medium is supplemented with the at least one compound after a period of 1 to 3 weeks.

15. The method of claim 1, wherein differentiating the neural spheroid into the hRNS of step (b) comprises:
(1) culturing the neural spheroid in suspension culture in neural medium comprising an inhibitor of GSK-3 and a sonic hedgehog pathway agonist for a period of 2 to 10 days;
(2) supplementing the neural medium with FGF4; and
(3) culturing the neural spheroid in suspension culture in neural medium comprising an inhibitor of GSK-3, sonic hedgehog pathway agonist and FGF4 for a period of 1 to 3 weeks.

16. The method of claim 15, further comprising following step (3):
(4) culturing the neural spheroid in suspension culture for a period of 2 to 10 days in neural medium comprising FGF4 and at least one of the compounds selected from the group consisting of: brain-derived neurotrophic factor (BDNF), NT3, L-Ascorbic Acid 2-phosphate Trisodium Salt (AA), N6, 2′-O-Dibutyryladenosine 3′, 5′-cyclic monophosphate sodium salt (cAMP), cis-4, 7, 10, 13, 16, 19-Docosahexaenoic acid (DHA), and DAPT; and
(5) culturing the neural spheroid in suspension culture for at least 1 week in neural medium comprising the at least one compound in the absence of FGF4.

17-18. (canceled)

19. The method of claim 1, wherein cells of the hRNS comprise at least one allele associated with a neurologic or psychiatric disorder, wherein the neurologic or psychiatric disorder is selected from the group consisting of: schizophrenia, affective disorder and autism spectrum disorder (ASD), optionally wherein the affective disorder is major depressive disorder, bipolar disorder or an anxiety disorder.

20-21. (canceled)

22. A human raphe nuclei spheroid (hRNS) obtained by the method of claim 1.

23. A method for producing a cortico-raphe nuclei assembloid (hCS-hRNS) in vitro, the method comprising:
(i) producing a human raphe nuclei spheroid (hRNS) by the method of claim 1 from a first human pluripotent stem cell
(ii) (a) inducing a second human pluripotent stem cell in a second suspension culture to a neural fate to derive a neural spheroid by culturing in a medium comprising an inhibitor of bone morphogenetic protein (BMP) and an inhibitor of transforming growth factor β (TGFβ), and wherein the method comprises supplementing the medium with an inhibitor of GSK-3 for a period of from 2 to 10 days;
(b) differentiating the neural spheroid into a cortical spheroid (hCS), and
(iii) culturing the hRNS and hCS under conditions permissive for cell fusion in neural medium, such that the cortico-raphe assembloid comprises human serotonergic neurons with projections between the hRNS and hCS, and neurons from the hCS projecting into hRNS wherein the human neurons form bidirectional projections between the hRNS and hCS.

24-38. (canceled)

39. The method of claim 23, wherein step (iii) comprises culturing the hRNS and hCS under conditions permissive for cell fusion for at least 3 days.

40. The method of claim 23, wherein step (iii) comprises culturing the hRNS and hCS in direct physical contact to each other.

41. The method of claim 23, wherein the suspension culture of step (i)(a) and/or step (ii)(a) is feeder layer free culture condition.

42. The method of claim 23, wherein cells of the cortico-raphe nuclei assembloid comprise at least one allele or genetic event associated with a neurologic or psychiatric disorder.

43. The method of claim 23, wherein either the first or second human pluripotent stem cell comprises at least one allele or genetic event associated with a neurologic or psychiatric disorder.

44. The method of claim 42, wherein the neurologic or psychiatric disorder is selected from the group consisting of: schizophrenia, affective disorder and autism spectrum disorder (ASD), optionally wherein the affective disorder is major depressive disorder, bipolar disorder or an anxiety disorder.

45. A cortico-raphe nuclei assembloid (hCS-hRNS) comprising a raphe nuclei spheroid (hRNS) fused to a cortical spheroid (hCS), wherein the hCS-hRNS comprises human serotonergic neurons projecting between the hRNS and hCS and forming a human neuromodulatory circuit, and wherein the hCS-hRNS is capable of being maintained in suspension culture without adhering to a surface for at least 4 weeks.

46. The cortico-raphe nuclei assembloid of claim 45, produced by the method of claim 23.

47. A method of determining the effect of a candidate agent on serotonergic modulation on cortical neural circuits, the method comprising: contacting the candidate agent with the cortico-raphe nuclei assembloid (hCS-hRNS) of claim 45; and determining the effect of the candidate agent on the ability of serotonergic neurons to modulate function of cortical neural circuits,
optionally wherein cells of the hCS-hRNS comprise at least one allele or genetic event associated with a neurologic or psychiatric disorder, further optionally wherein the neurologic or psychiatric disorder is selected from the group consisting of: schizophrenia, affective disorder and autism spectrum disorder (ASD).

48. The method of claim 47, wherein the candidate agent is a selective serotonin reuptake inhibitor (SSRI).
